(19) United States
(12) Patent Application Publication
(10) Pub. No.: US 2017/0044476 A1
(43) Pub. Date: Feb. 16, 2017

(54) CULTURE DISH

(71) Applicant: Unisense FertiliTech A/S, Aarhus N (DK)

(72) Inventors: Kim Bondergaard Madsen, Aarhus C (DK); Jonas Lerche Hansen, Hinnercup (DK); Soren Porsgaard, Aarhus N (DK); Niels Ramsing, Risskov (DK); Jorgen Bertsen, Viborg (DK)

(73) Assignee: Unisense FertiliTech A/S, Aarhus N (DK)

(21) Appl. No.: 15/306,489

(22) PCT Filed: Mar. 24, 2015
(86) PCT No.: PCT/EP2015/056302

§ 371 Date: Oct. 24, 2016

(30) Foreign Application Priority Data
May 7, 2014 (GB) 1408039.4

(51) Int. Cl. C12M 1/32; C12M 1/34; C12M 3/00

(52) U.S. Cl. CPC C12M 23/12; C12M 21/06; C12M 41/36

(57) ABSTRACT

A culture dish for holding one or more object to be cultured is described. The culture dish has a main body comprising at least one well for receiving an object to be cultured and a quantity of culturing media for the object, such as a water-based growth media, and a reservoir for receiving a quantity of cover media, such as mineral oil. The at least one well is provided in a floor of the reservoir so that when in normal use cover media in the reservoir overlays culturing media in the at least one well. The reservoir is defined by the reservoir floor and a reservoir wall extending away from the reservoir floor. At least a section of the reservoir wall is angled away from the vertical so as to be inclined with respect to a horizontal plane defined by a surface of cover media in the reservoir when the culture dish is in normal use. The angled section of the reservoir wall can help reduce the appearance of a meniscus in the cover media overlaying the wells, and can furthermore be positioned so as to provide a ready indication of when the reservoir contains an appropriate level of cover media for culturing.

### BACKGROUND OF THE INVENTION

The present invention relates to culture dishes. More particularly, certain embodiments relate to culture dishes for incubating embryos.

Infertility affects more than 80 million people worldwide. It is estimated that 10% of all couples experience primary or secondary infertility. In vitro fertilization (IVF) is an elective medical treatment that may provide a couple who has been otherwise unable to conceive a chance to establish a pregnancy. It is a process in which eggs (oocytes) are taken from a woman's ovaries and then fertilized with sperm in the laboratory. The embryos created in this process are then placed into the uterus for potential implantation. In between fertilization (insemination) and transfer the embryos are typically stored in an incubation chamber of an incubator for 2-6 days during which time they may be regularly monitored, for example through imaging, to assess their development. Conditions within the incubator, such as temperature and atmospheric composition, are controlled, generally with a view to emulating the conditions in the oviduct and uterus.

Embryos for incubation are typically placed in culture dishes, which may then be stored in the incubator. Culture dishes may also referred to as slides, carriers or trays.

One well-known apparatus for incubating embryos, and which also provides for time-lapse embryo imaging to assess embryo development, is the EmbryoScope® device with its associated EmbryoViewer® software developed by, and available from, Unisense FertiliTech A/S (Aarhus, Denmark). The EmbryoScope® apparatus has the ability to incubate embryos on six removable slides supported by a slide carrier. Each slide (dish) comprises a 3×4 array of receptacles and so is able to hold up to 12 embryos. In use, each embryo to be incubated is placed in a separate receptacle in its own media droplet separate from the others. The EmbryoScope® apparatus has a built-in microscope and translation stage to allow the embryos to be sequentially imaged at different stages throughout their incubation.

FIG. 1 is a schematic perspective view of an embryo dish/slide 2 of the kind typically used in the EmbryoScope® device. The dish 2 has overall dimensions of around 7.5 cm (length)×2.5 cm (width)×1.5 cm (height) and is formed as a single injection moulding of a plastics material, for example a transparent polyester material. The dish 2 comprises a main body 4, a handle 6 for holding the dish, and a labelling area 8 on which a label may be stuck with information relating to the embryos on the slide (e.g. patient ID and incubation protocol information). A 3×4 array of receptacles (wells) 10 for receiving individual embryos for culturing are provided within a recess 12 in the main body 4. The recess 12 is defined by a recess floor 14, in which the receptacles 10 are provided and recess walls 16. The recess has dimensions of around 3.5 cm (length)×2.0 cm (width)×0.8 cm (depth). The normal orientation for the slide 2 during use is with the recess floor 14 horizontal and the recess walls 16 vertical. The receptacles 10 have a diameter of around 3.5 mm at the recess floor and have vertical walls extending downwards from the recess floor for around 2.5 mm before tapering to a smaller sub-millimetre (e.g. around 0.2 mm diameter) well 18 in which an embryo is located for culturing. Within the recess 12 there is also provided four (two at each end) flush reservoirs 20. These may be used to store liquids, for example cleaning liquids, used while the embryos are prepared for culturing/incubation in accordance with whichever protocols are being followed. Although not shown in FIG. 1, the slide 2 has a separate lid that may be placed over a portion of the main body 4 containing the recess 12.

In normal use individual embryos are located in respective ones of the sub-millimetre wells 18 at the bottom of the receptacles 10. The number of wells 10 containing an embryo on any given slide will depend on the number of embryos to be incubated using that slide. It is common to avoid mixing embryos from different patients on the same side, and so if there are not enough embryos from a patient to fill a complete slide, the remaining receptacles for the slide will generally remain unused. Each receptacle 10 containing an embryo is filled (to a level below the recess floor 14) with a water-based culturing media for the embryo. The recess 12 is then at least partially filled with an oil layer that overlays the culturing (growth) media in the receptacles 10. The oil layer provides a barrier to help reduce evaporation of the culturing media in which the embryos are located.

The geometry and dimensions of the dish 2 represented in FIG. 1 are adapted to match those of the specific apparatus in which the embryos are to be incubated using the slide. However, broadly corresponding designs of culture dish/embryo slide may be used for other incubator/culturing apparatus.

More details on the characteristics of known culture dishes suitable for use in embryo incubation can be found, for example, in WO 09/003487 (Unisense Fetilitech A/S) [1] and WO 01/002539 (The Danish Institute of Agricultural Sciences) [2].

While culture dishes of the kind represented in FIG. 1 have been found to be successful in facilitating embryo incubation, and in particular in the context of time-lapse imaging systems, the present inventors have nonetheless recognised there are still some aspects of the design which could be improved.

### SUMMARY OF THE INVENTION

According to a first aspect of the invention there is provided a culture dish for holding one or more object to be cultured, wherein the culture dish has a main body comprising: at least one well for receiving an object to be cultured and a quantity of culturing media for the object; and; a reservoir for receiving a quantity of cover media, wherein the at least one well is provided in a floor of the reservoir so that when in normal use cover media in the reservoir overlays culturing media in the at least one well, wherein the reservoir is defined by the reservoir floor and a reservoir wall extending away from the reservoir floor, and wherein at least a section of the reservoir wall is angled away from the vertical so as to be inclined with respect to a horizontal plane defined by a surface of cover media in the reservoir when the culture dish is in normal use.

In accordance with some embodiments the angled section of the reservoir wall extends all around the reservoir.

In accordance with some embodiments the angled section of the reservoir wall is inclined with respect to the horizontal plane by an angle within a range selected from the group comprising: 10 degrees to 80 degrees; 20 degrees to 70 degrees; 30 degrees to 60 degrees; and 40 degrees to 50 degrees.

In accordance with some embodiments the angled section of the reservoir wall is located above a lower section of the reservoir wall, and wherein the angled section and the lower section are at different angles to the horizontal plane.

In accordance with some embodiments the height of the interface between the lower section of the reservoir wall and the angled section of the reservoir wall above the reservoir floor is selected according to a minimum level for cover media to be used when the culture dish is in normal use.

In accordance with some embodiments the angled section of the reservoir wall is located below an upper section of the reservoir wall, and wherein the angled section and the upper section are at different angles to the horizontal plane.

In accordance with some embodiments the upper section of the reservoir wall defines a section of the reservoir wall extending above a surface of the main body around the reservoir.

In accordance with some embodiments the culture dish further comprises a depression in the floor of the reservoir, wherein the depression is defined by a depression floor and a depression wall, and wherein the at least one well is provided in the depression floor.

In accordance with some embodiments at least a section of the depression wall is angled away from the vertical so as to be inclined with respect to the horizontal plane.

In accordance with some embodiments the angled section of the depression wall extends all around the depression.

In accordance with some embodiments the angled section of the depression wall is adjacent the depression floor at the bottom of the depression wall.

In accordance with some embodiments the angled section of the depression wall is inclined with respect to the horizontal plane by an angle within a range selected from the group comprising: 10 degrees to 80 degrees; 20 degrees to 70 degrees; 30 degrees to 60 degrees; and 40 degrees to 50 degrees.

In accordance with some embodiments the depression floor includes a raised section of floor that is higher than the surrounding depression floor.

In accordance with some embodiments a portion of the depression wall is recessed so that is further from the at least one well than parts of the depression wall adjacent the recessed portion.

In accordance with some embodiments the at least one well comprises a plurality of wells provided in the depression floor of the depression.

In accordance with some embodiments the at least one well comprises a plurality of wells and the culture dish comprises at least one further depression in the floor of the reservoir, and wherein different wells are provided in different depressions.

In accordance with some embodiments the at least one well comprises an upper well section and a lower well section separated by a shelf section.

In accordance with some embodiments at least an upper portion of the at least one well is non-circular in horizontal cross-section.

In accordance with some embodiments the at least one well comprises a plurality of wells.

In accordance with some embodiments the culture dish further comprises a pair of fins extending away from the main body to provide a handle for the culture dish.

In accordance with some embodiments the culture dish further comprises a labelling region arranged on the main body between the pair of fins for receiving a label containing information relating to the culture dish.

In accordance with some embodiments the labelling region is inclined relative to the horizontal plane.

In accordance with some embodiments the culture dish further comprises a removable lid for covering the reservoir.

In accordance with some embodiments an upper surface of the removable lid comprises an upwardly extending lip at its periphery.

In accordance with some embodiments the lid does not overhang the main body when covering the reservoir.

In accordance with some embodiments the culture dish further comprises at least one receptacle for media to be used for preparing objects for culturing.

In accordance with some embodiments the at least one receptacle is within the reservoir.

In accordance with some embodiments at least a section of an inner wall of the least one receptacle is inclined with respect to the horizontal plane.

In accordance with some embodiments the culture dish further comprises an annotation area having a surface texture which is different from a surface texture of other parts of the culture dish.

In accordance with some embodiments the at least one well comprises a plurality of wells arranged along an arc of a circle.

In accordance with some embodiments the plurality of wells are spatially arranged in groups, wherein neighbouring wells that are in different groups are separated by a greater distance than neighbouring wells that are in the same groups.

According to a second aspect of the invention there is provided an incubator apparatus comprising an incubation chamber comprising at least one culture dish according to the first aspect of the invention.

According to a third aspect of the invention there is provided a method of culturing at least one object, the method comprising: providing a culture dish having a main body comprising: at least one well for receiving an object to be cultured and a quantity of culturing media for the object; and; a reservoir for receiving a quantity of cover media, wherein the at least one well is provided in a floor of the reservoir so that when in normal use cover media in the reservoir overlays culturing media in the at least one well, wherein the reservoir is defined by the reservoir floor and a reservoir wall extending away from the reservoir floor, and wherein at least a section of the reservoir wall is angled away from the vertical so as to be inclined with respect to a horizontal plane defined by a surface of cover media in the reservoir when the culture dish is in normal use; and wherein the method further comprises filling the reservoir with cover media to a level that meets the section of the reservoir wall is angled away from the vertical.

It will be appreciated that features and aspects of the invention described above in relation to the first and other aspects of the invention are equally applicable to, and may be combined with, embodiments of the invention according to other aspects of the invention as appropriate, and not just in the specific combinations described above.

### BRIEF DESCRIPTION OF THE DRAWINGS

The invention is now described by way of example only with reference to the following drawings in which:

FIG. 1 schematically represents in perspective view a known culture dish to be used for incubating embryos;

FIGS. 2 to 13 schematically represent different views of a culture dish in accordance with an embodiment of the invention;

FIG. 14 schematically represents in perspective view an integrated apparatus for use in conjunction with the culture dish represented in FIGS. 2 to 13;

FIGS. 15 and 16 schematically represent different views of the culture dish represented in FIGS. 2 to 13 in position in a slide carrier of the incubator apparatus represented in FIG. 14;

FIGS. 17 and 18 schematically represent features of culture dishes according to certain further embodiments of the invention;

FIGS. 19 to 21 schematically represent different views of a culture dish in accordance with another embodiment of the invention; and

FIG. 22 schematically represents a plan view of a culture dish in accordance with another embodiment of the invention.

### DETAILED DESCRIPTION

Aspects and features of certain examples and embodiments of the present invention are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with conventional techniques for implementing such aspects and features.

Unless the context demands otherwise, the terms used herein should be interpreted in accordance with their meanings as commonly understood by one of ordinary skill in the art to which this disclosure belongs.

Embryos are typically incubated for a period of up to 3 to 5 days following fertilisation. In some respects the term “embryo” may sometimes be used to refer to a fertilised oocyte (egg) after implantation in the uterus until 8 weeks after fertilization, at which stage it become a fetus. In accordance with this terminology the fertilized oocyte may be sometimes called a pre-embryo or zygote until implantation occurs. However, for convenience the term “embryo” may sometimes also be used to encompass the zygote stage prior to implantation, and this approach will generally be followed herein. That is to say, the term “embryo” is used herein in a broad sense to cover all developmental stages from fertilization of an oocyte through morula, blastocyst stages, hatching and implantation. Accordingly, the term embryo may be used herein to denote each of the stages: fertilized oocyte, 2-cell, 4-cell, 8-cell, 16-cell, compaction, morula, blastocyst, expanded blastocyst and hatched blastocyst, as well as all stages in between (e.g. 3-cell or 5-cell) stages. Thus, the terms embryo and zygote may be used herein interchangeably, for example. An embryo that is incubated using a culture dish in accordance with embodiments of the invention such as described herein may be previously frozen, e.g. embryos cryopreserved immediately after fertilization (e.g. at the 1-cell stage) and then thawed. Alternatively, they may be freshly prepared, e.g. embryos that are freshly prepared from oocytes by IVF or ICSI techniques for example.

An embryo is approximately spherical and is composed of one or more cells (blastomeres) surrounded by a gelatine-like shell, the acellular matrix known as the zona pellucida. The zona pellucida performs a variety of functions until the embryo hatches, and is a good landmark for embryo evaluation. The zona pellucida is spherical and translucent, and should be clearly distinguishable from cellular debris.

As noted above, embryos are sometimes stored/held in a culture dish, for example during in vitro fertilization (IVF) procedures. In this context a culture dish may also referred to as an (embryo) slide, (embryo) carrier or (embryo) tray. As also noted above, a culture dish for use in embryology will typically comprise a plurality of wells for receiving embryos to be cultured. Embryos in respective wells are submerged in a water-based growth media (culturing media) which is overlaid by a layer of oil layer.

An institute using embryo culture dishes may be dealing with many embryos from many patients at the same time. This means it important for culture dishes to be convenient to use and handle. However, it is also desirable for culture dishes to be designed in a way which aims to help a user follow the general aims of good laboratory practice, for example in terms of reducing the risk of errors in handling (e.g. damaging or mixing-up embryos), minimising the usage of consumables (such as oil), and facilitating analyses to be applied to the embryos (for example time lapse imaging). With this in mind, the inventors have conceived of new configurations of embryo dish, for example for incubating embryos in an incubator, such as an incubator in an apparatus that provides for time-lapse imaging of embryos.

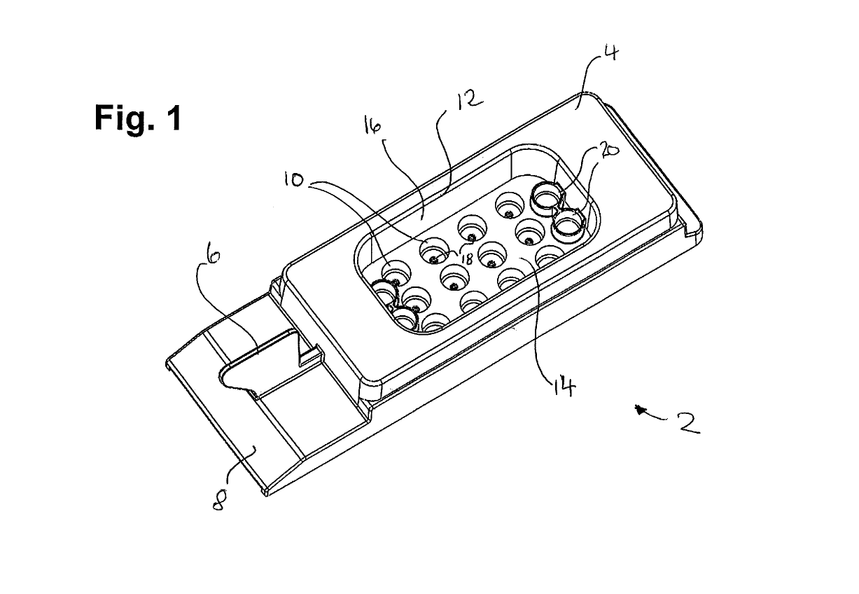

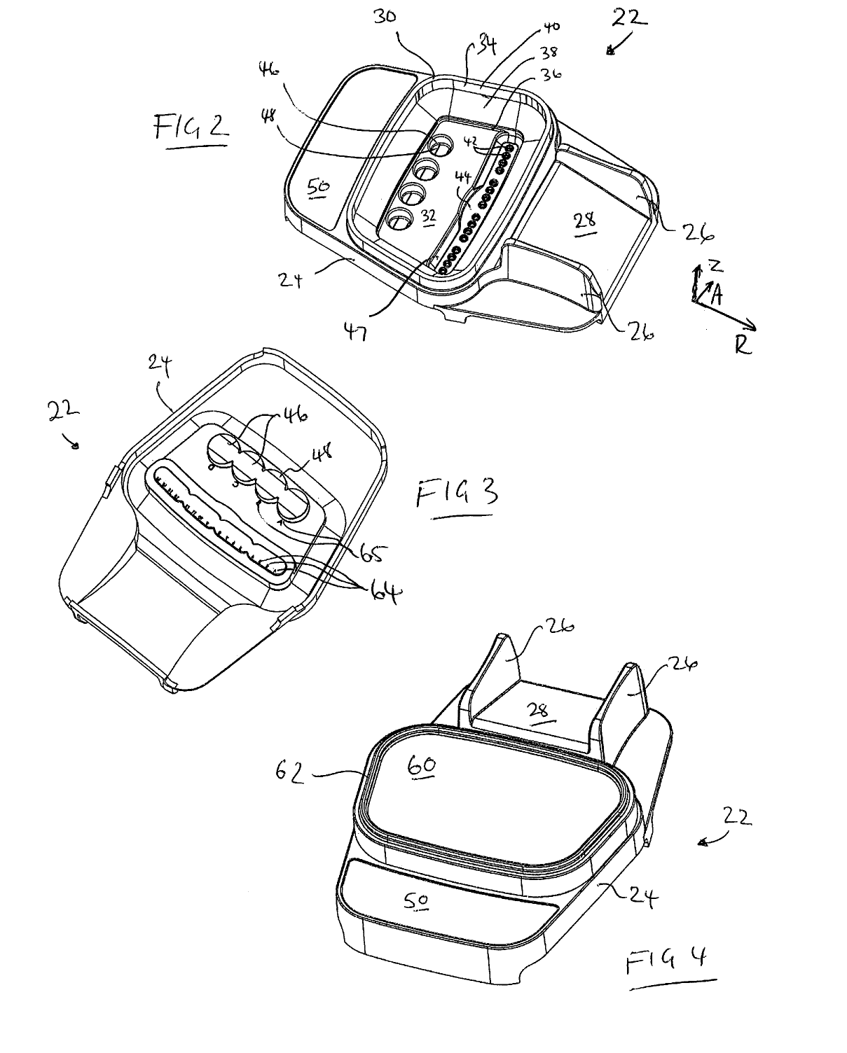

FIGS. 2 to 13 schematically represent a culture dish 22 according to certain embodiments of the invention. The culture dish 22 comprises a main body 24 which may be manufactured in accordance with conventional techniques, for example injection moulding of a suitable plastics material. What is significant for certain embodiments of the invention is not the specific techniques used in manufacturing the culture dish, but the shape and configuration of the culture dish. In this regard the manufacturing techniques and materials used for the culture dish 22 may be generally the same as those used for known culture dishes, such as the culture dish 2 represented in FIG. 1 and discussed above. In particular, the culture dish 22 may be formed by injection moulding of a generally transparent polymer, for example a polyester, such as PEN, PETg, and/or PET. The main body 24 may comprise a single moulding. As discussed further below, the culture dish 22 may in some cases further comprise a lid which is separate and removable from the main body 24. The lid may again be manufactured in accordance with conventional techniques, for example injection moulding of a transparent polymer material.

Before discussing particular features and aspects of the culture dish (slide/tray/carrier) 22 represented in FIGS. 2 to 13, an overall summary of the different figures is provided.

FIG. 2 schematically represents the culture dish 22, and in particular the main body 24 of the culture dish 22, in perspective view from above.

FIG. 3 schematically represents the culture dish 22, in perspective view from below. The underside of various features of the culture dish 22 seen from above in FIG. 2 can be seen in FIG. 3. This shows the main body 24 is generally sheet-like rather than solid, as is also apparent in some of the cross-section views discussed further below. The cross-sectional thickness of the material comprising the main body 24 may be generally around 1 or 2 mm, but may be thicker or thinner in different places according to the generally understood principles of construction for injection moulding of culture dishes.

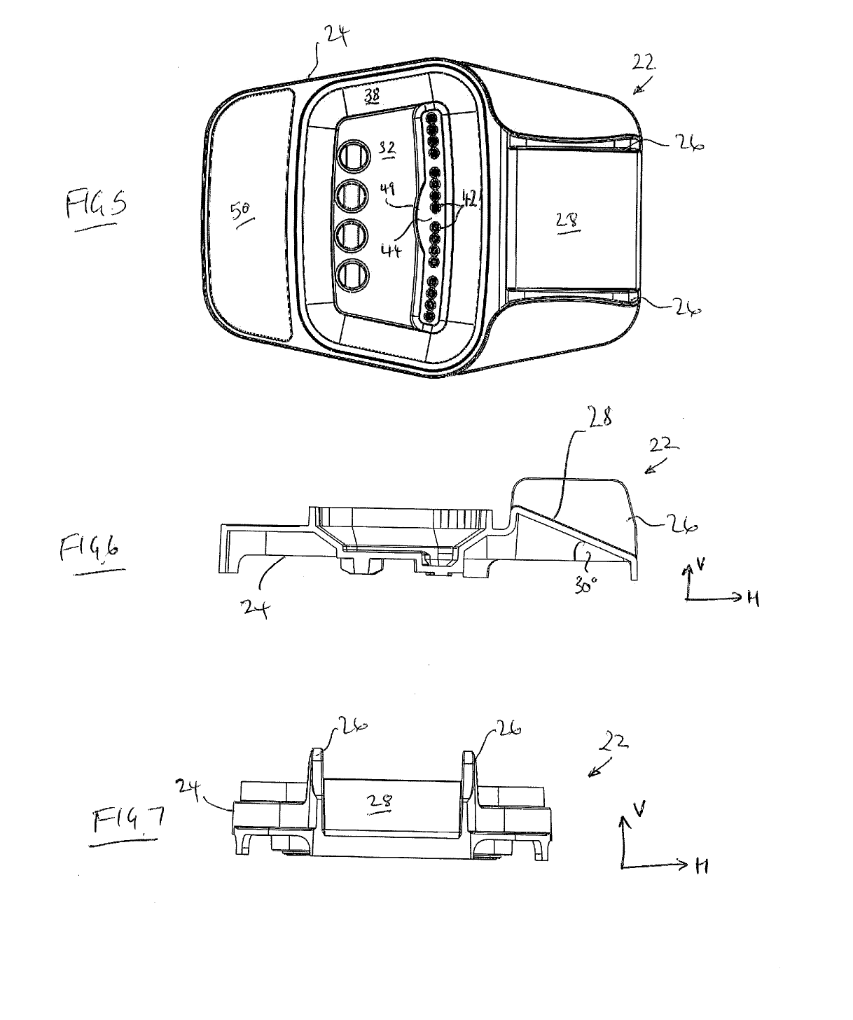

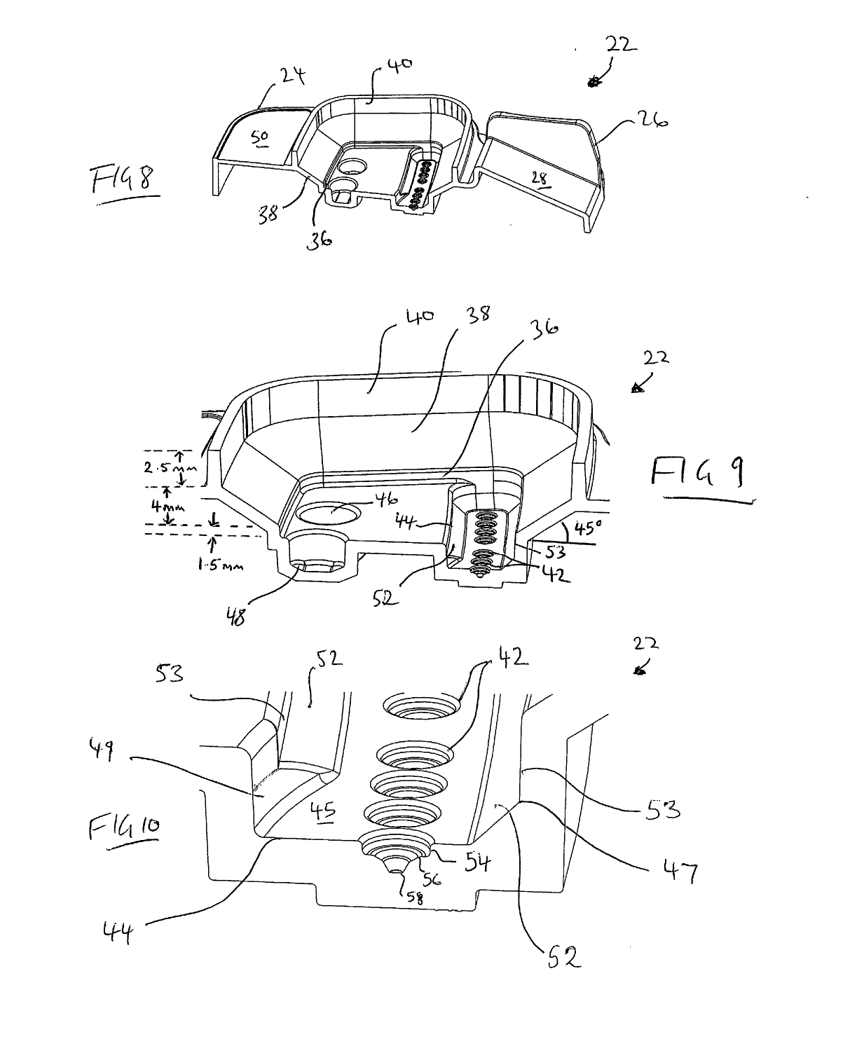

FIG. 4 schematically represents the culture dish 22 in a perspective view from above and from a different direction to that represented in FIG. 2. Furthermore, the culture dish 22 as represented in FIG. 4 is shown with a lid 60 covering a part of the main body 24. As noted above, the lid may be manufactured in accordance with conventional techniques, for example injection moulding of a transparent polymer material.

FIG. 5 schematically represents the culture dish 22 in plan view from above.

FIG. 6 schematically represents a cut-away side view of the culture dish 22 for a cut running along that is horizontal and through the middle of the culture dish 22 as represented in FIG. 5.

FIG. 7 schematically represents an end-view of the culture dish 22 as seen from the right-hand side for the orientation represented in FIG. 5.

FIG. 8 schematically represents a cut-away perspective view of the culture dish 22.

FIGS. 9 and 10 schematically represent cut-away perspective views of certain parts of the culture dish 22.

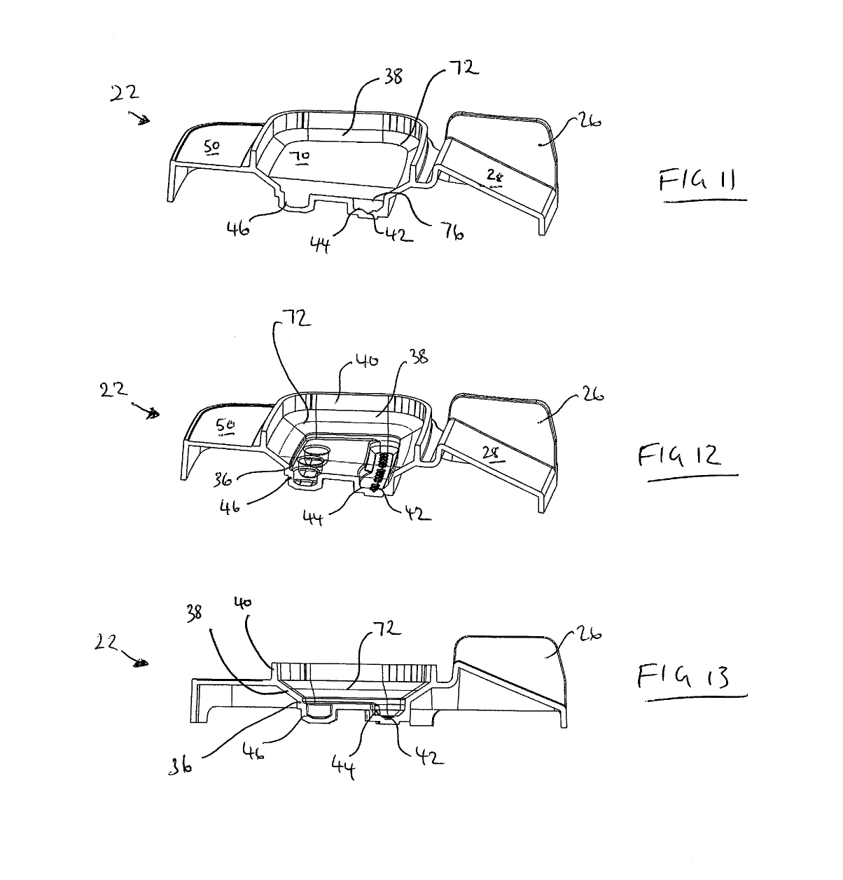

FIGS. 11 and 12 schematically represents cut-away perspective views of the culture dish 22 similar to the view represented in FIG. 8, but showing the culture dish containing culturing media and cover media (oil). In FIG. 11 the culturing media and cover media in the culture dish is shown as solid white to more clearly represented surface, whereas in FIG. 12 the culturing media and cover media is shown as transparent.

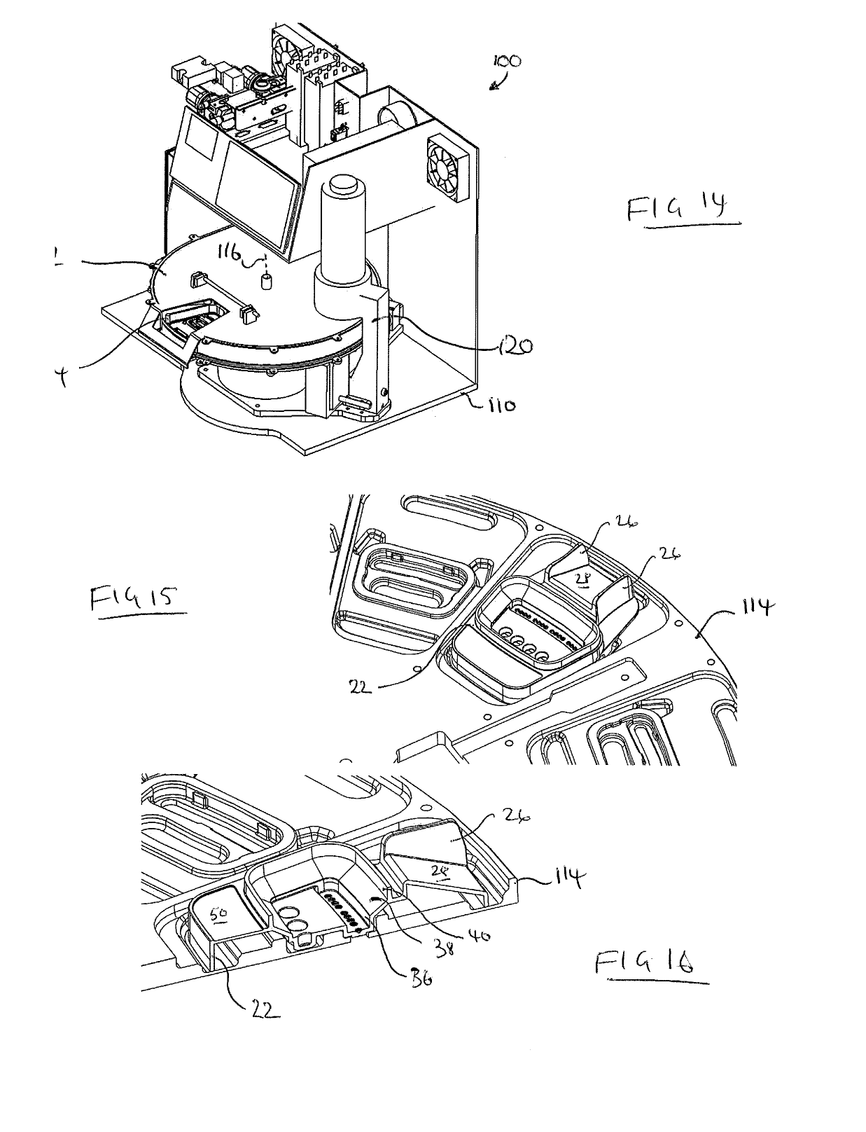

FIG. 13 schematically represents a cut-away side view of the culture dish 22 similar to the view represented in FIG. 6 (although with a slightly different plane of cut), and showing the culture dish containing media (including embryo culturing media and an overlying layer of cover media).

The culture dish 22 represented in FIGS. 2 to 13 in this particular example is intended for use in an incubator apparatus 100 such as schematically represented in perspective view in FIG. 14. The incubator apparatus 100 represented in FIG. 14 may, for example, be of the kind described in co-pending UK patent applications GB 1401773.5 [3] (filed 3 Feb. 2014) and/or GB 1401774.3 [4] (filed 3 Feb. 2014). However, it will be appreciated the specific incubator apparatus to be used for a culture dish according to embodiments of the invention (if indeed the culture dish is to be used for incubating embryos) is not overly significant.

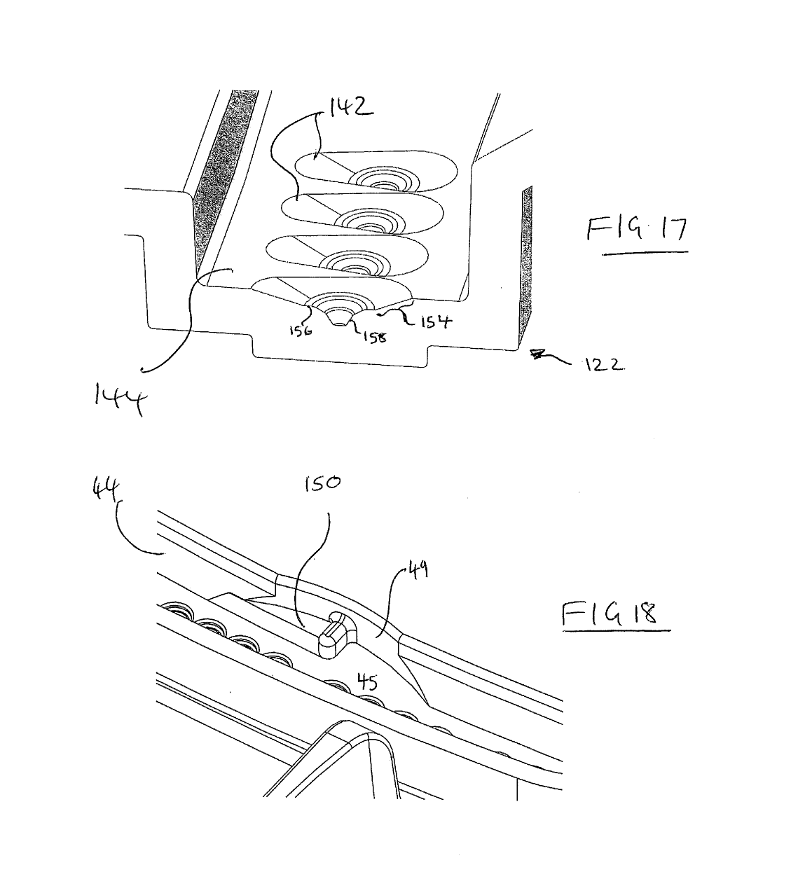

The incubator apparatus 100 in this example has a characteristic footprint on the order of 60 cm×50 cm and a height that is on the order of 50 cm. The apparatus 100 comprises an outer casing which is not shown in FIG. 14 so as to reveal various internal components of the incubator apparatus. The apparatus 100 comprises a base plate 110 to which various other components are mounted. At its heart the incubator 100 includes an incubation chamber defined by an incubation chamber housing 112 and a slide carrier 114. The slide carrier 114 comprises a plurality of compartments for holding respective embryo culture dishes of the kind represented in FIGS. 2 to 13 for holding embryos to be incubated within the incubation chamber. The slide carrier 114 is generally in the form of a circular disc, although only a small part of the slide carrier 114 is visible in FIG. 14. A larger portion of the slide carrier 114 is shown in schematic perspective view in FIG. 15 without the surrounding incubator chamber housing 112 and with a culture dish 22 located in one of the slide carrier's compartments. FIG. 16 is similar to FIG. 15, but shows a partial perspective cut-away view through the culture dish 22 and slide carrier 114.

The incubator apparatus 100 further comprises an imaging device 120, in this case a digital microscope. The microscope 120 is mounted outside the incubation chamber in alignment with a viewing port in the incubation chamber housing 112 to allow the microscope to record images of embryos stored in culture dishes.

The incubation chamber housing 112 and the slide carrier 114 are both generally circular and relatively thin (i.e. disc like). The incubation chamber housing 112 is fixed in position relative to the base plate 110. The slide carrier 114 is rotatable within the incubation chamber defined by the incubation chamber housing 112 about a rotation axis 116. In this example the slide carrier 114 is mounted directly to a shaft of a motor mounted below, and outside, the incubation chamber. Thus the shaft of the motor passes through an opening in the underside of the incubation chamber and is coupled to the slide carrier such that the motor can drive the slide carrier to rotate within the incubation chamber. Thus different culture dishes within the incubation chamber may be rotated into alignment with the imaging device for monitoring (image acquisition).

Overall, the operation and construction of the incubator apparatus 100 may follow known techniques, such as those described in UK patent applications GB 1401773.5 [3] and GB 1401774.3 [4].

Thus, in normal use, a culture dish 22 according to an embodiment of the invention may be placed in a compartment of a slide carrier 114 of an incubator apparatus 100 of the kind represented in FIG. 14. The relative spatial arrangement of features of the culture dish 22 may be described with reference to its orientation during normal use.

Thus, the term horizontal may be used to describe a plane of the culture dish 22 as represented in FIG. 5, which in this example is generally the plane in which the culture dish 22 has its greatest areal extent. The term vertical may be used to describe a direction which is normal to the horizontal. Thus, the direction which may be referred to as a vertical direction for the culture dish is as schematically represented by direction arrow marked V in FIGS. 6 and 7. Directions referred to herein as horizontal directions for the culture dish are directions which are parallel to the plane of FIG. 5, for example as schematically represented by the direction arrows marked H in FIGS. 6 and 7. The vertical direction may also be referred to as the Z-direction for the culture dish 22. Because the culture dish 22 in this example is intended for use in an incubator in which culture dishes are rotated about an axis for sequentially aligning embryos with an imaging system, it can be convenient in some cases for directions in the horizontal plane of the culture dish 22 to be referred to within a circular coordinate system having its origin at the centre of rotation 116 of the slide carrier, a radial direction R extending away from the centre of rotation and an azimuthal direction A extending perpendicular to the radial direction. Thus the relative arrangement of features of the culture dish 22 may in some cases be described by reference to a radial direction R, an azimuthal direction A and a vertical direction Z, as schematically indicated in FIG. 2. The radial direction R may also be referred to as an axis of extent/length direction L for the culture dish. A width direction W for the culture dish may be defined as a direction which is horizontal and orthogonal to the length direction L. A height direction H for the culture dish may be defined as a direction which is vertical. Of course it will be appreciated these various directions are defined purely for the convenience in explaining the relative arrangement of some features of the culture dish, and in particular having regard to an orientation of the culture dish when in normal use and the terms are not intended to in themselves impose any particular structural limitations on the overall configuration of the culture dish 22 in an absolute sense.

Terminology such as “up” and “down” and “top” and “bottom” will be used herein having regard to the vertical direction for the slide when in normal use. Thus the “top” of the culture dish is the surface of the culture dish which faces upwards when the culture dish is in normal use, for example when containing embryos and media. The “bottom” of the culture dish is the surface of the culture dish which faces downwards when the culture dish is in normal use. The edge surfaces of the main body of the culture dish which are generally orthogonal to its axis of extent may be referred to as the ends of the culture dish. The edge surfaces of the main body of the culture dish which are generally parallel to its axis of extent may be referred to as the sides of the culture dish.

As is apparent from the figures, the ends of the culture dish 22 in this example are generally straight while the sides are bent broadly around their middles so the sides taper inwards. This tapering allows multiple culture dishes to be conveniently arranged around a circle when placed in a slide carrier 114 of the kind represented in FIGS. 15 and 16.

The bends in the sides of the culture dish and the outer corners between the ends and the sides are rounded. The culture dish 22 in this particular example has a characteristic length L of around 6.5 cm, a characteristic width W (at the widest point) of around 5 cm or so, and characteristic height H of around 1.5 cm. However, it will be appreciated that other sizes and shapes of culture dish may be selected according to the implementation at hand, for example to match the geometry of a holder for the culture dishes. For example, in accordance with various embodiments of the invention, and according to implementation, a culture dish may have a characteristic length L within a range selected from the group comprising: 2 cm to 20 cm; 2 cm to 15 cm; 3 cm to 12 cm and 5 cm to 10 cm; and/or a characteristic width W within a range selected from the group comprising: 1 cm to 10 cm; 2 cm to 8 cm; 3 cm to 7 cm and 4 cm to 6 cm; and/or a characteristic height H less than or equal to an amount selected from the group comprising: 2 cm; 1.5 cm; 1.0 cm; and 0.5 cm. However, It will be appreciated the overall scale of the culture dish is not of primary significance and may be selected according to the implementation at hand, for example having regard to an intended use.

Referring to FIGS. 2 to 13, the culture dish 22 is for holding one or more objects to be cultured, such as embryos, and comprises a main body 24, as discussed above.

The main body comprises sixteen wells 42 for receiving embryos for culturing. In normal use an embryo culturing media, for example a water-based nutrient rich media, is also placed in the wells with the embryos. In use, there will typically be one embryo in each well up to the number of embryos to be cultured. For example if there are nine embryos from a patient to be cultured using a given culture dish, there will be one embryo placed in each of nine of the wells 42 with no embryos in the remaining seven wells. That is to say, it would be uncommon, at least for human IVF, to simultaneously populate all the wells of a particular culture dish by mixing embryos from different patients on the same culture dish/slide. Whilst there might typically be one embryo in each well, in some applications there may be a desire for multiple embryos to be inspected as a good culture in a single well. Accordingly, in some situations an individual well may comprise multiple embryos.

The sixteen wells 42 in this example are spatially arranged along a single line comprising an arc of a circle. The radius of this arc is selected according to the separation between the wells 42 and the axis of rotation 116 of the slide carrier 114 when the culture dish 22 is located for use in the slide carrier 114, such as represented FIG. 15. This can allow a monitoring station of the incubation apparatus 100, for example the imaging device 120, to be brought into alignment with different wells 42 by simply rotating the slide carrier on which the culture dish 22 is held to an appropriate location. In one example, an arc radius of 140 mm might be used, but it will be appreciated the appropriate arc radius will depend on the geometry of the incubator.

The sixteen wells 42 in this example are spatially arranged into four groups of four. The distance between neighbouring pairs of wells within a group is less than the distance between neighbouring wells at the ends of different groups. This spatial separation of wells into a number of different groups has been found to help a user more readily locate a particular well. For example, if the user wishes to identify the tenth well from one side of the sample carrier, the user can simply look to the second well in the third group, rather than having to count along the wells from one side of the culture dish. The wells in this example are also provided with identification markings 64 to assist a user identify the different wells. The identification markings 64 may be moulded into the main body of the culture dish adjacent the respective wells, and may, for example, comprise a numeric index from 1 to 16. Such markings may be moulded into the underside of the culture dish so they are still visible from above, but do not introduce surface roughness which could potentially trap contaminants in the vicinity of the wells. The identification markings 64 can be seen, for example, on the underside perspective view represented in FIG. 3. (Corresponding identification markings 65, in this case comprising the letters A, B, C, D, for the flush reservoirs 46 discussed further below can also be seen in the underside view of FIG. 3).

The wells 42 are, in this example, generally circularly symmetric about a vertical axis and have a depth of around 2.5 mm. The centres of neighbouring wells within a group are separated by around 1.75 mm. Of course other separations may be used in other implementations. The centres of neighbouring wells at the ends of adjacent groups of wells are separated by around twice this amount, i.e. 3.5 mm. As can be seen in FIG. 10, each well 42 comprises an upper well section 54 and a lower well section 58 separated by a generally horizontal shelf section 56—i.e. the horizontal cross section at the top of the lower well section is less than the horizontal cross section at the bottom of the upper well section.

The upper well section 54 is generally in the form of a circular cylinder (i.e. having vertical walls). The lower well section 58 is generally in the form of a truncated circular cone that narrows with increasing depth down to a well floor at the base of the well 42, which is generally where an embryo will rest during culturing. The characteristic width/diameter of the well at its base may be sub-millimetre, for example around 0.25 mm or so. In this example the upper well section has a height of around 1.0 mm and the lower well section has a height of around 1.5 mm.

The shelf section 56 is provided to help prevent particulate contaminants in suspension in liquid media within the culture dish from settling to the floor of the wells 42. A fraction of particulate contaminants which settle into a well will settle on the shelf section 56, rather than on the floor of the wells at the bottom of the lower well section 58. The larger the relative size of the shelf section 56, the greater the fraction of particulate contaminants that will be prevented from settling on the floor of the well 42. The shelf section in this example has a width in the horizontal plane of around 0.5 mm. The majority of the shelf section 56 may be horizontal. However, in other cases the shelf section 56 may be angled away from the horizontal, for example downwards away from the centre of the well so any settling particles are biased away from the centre of the well. As can be seen in FIG. 10, the transitions between the different sections of the well are generally rounded. Providing smooth/rounded transitions between different sections of the culture dish can help during manufacturing by injection moulding and can also reduce the potential for contaminants and/or bubbles becoming trapped in corners. The rounded corners and transitions can also help an object being cultured, such as an embryo, to settle at the bottom of the well 42.

The culture dish 22 comprises a reservoir 30 defined by a reservoir wall 34 and a reservoir floor 32. The wells 42 are provided in the floor 32 of the reservoir 30. More particularly, in this example implementation the wells 42 are provided within a depression (trough) 44 provided in the reservoir floor 32. All the wells are in the floor of a single trough 44, but in other examples the wells may be grouped together in different troughs in the reservoir floor 32.

The reservoir is for holding a quantity of cover media over the embryos and their culturing media when the culture dish is in use. The use of a cover media follows generally conventional culturing techniques whereby culturing media in wells containing embryos are overlaid by a layer of cover media, e.g. an oil-based media that is lighter than, and does not mix with, the culturing media, e.g. mineral oil. The cover media provides a barrier between the culturing media and the environment surrounding the culture dish, for example to help prevent evaporation of the culturing media. FIGS. 11 to 13 schematically represent the culture dish 22 when containing a quantity of cover media 70. The cover media 70 (e.g. mineral oil) will in general be transparent, but is represented as solid white in FIG. 11 to aid representation. In FIGS. 12 and 13 the cover media is represented as being transparent such that only its upper surface 72 is apparent.

In horizontal cross section the reservoir 30 has a generally quadrangular form with rounded corners. Accordingly, the reservoir wall 34 comprises two sides running broadly parallel to the ends of the culture dish 22 and two sides running broadly parallel to the sides of the culture dish 22. Because the sides of the culture dish 22 are angled with respect to one another, the sections of the reservoir wall 34 running broadly parallel to the sides of the culture dish 22 are also angled with respect to another.

The top of the reservoir in this example has a characteristic extent of around 3 cm between the sides running parallel to the ends of the culture dish 22 and a characteristic width between the sides running parallel to the sides of the culture dish of around 4 cm (at the widest point). The reservoir has a characteristic depth (from the top of the reservoir wall 34 to the reservoir floor 32) of around 0.8 cm. However, it will be appreciated that other sizes and shapes of reservoir may be selected according to the implementation at hand. For example, in accordance with various embodiments of the invention, and according to implementation, a reservoir may have a characteristic extent along the radial direction within a range selected from the group comprising: 1 cm to 5 cm; 2 cm to 4 cm and 2.5 cm to 3.5 cm; and/or a characteristic width within a range selected from the group comprising: 1 cm to 8 cm; 3 cm to 7 cm and 4 cm to 6 cm; and/or a characteristic height H less than or equal to an amount selected from the group comprising: 2 cm; 1.5 cm; 1.0 cm and 0.5 cm. However, It will be appreciated the overall scale and exact geometry of the reservoir in horizontal cross-section is not of primary significance and may be selected according to the implementation at hand, for example having regard to the intended use (e.g. a desired amount of cover media to be used).

The reservoir wall 34 extends upwardly from the reservoir floor 32 and in this example comprises three sections, each of which extends all around the reservoir 30. Thus, the reservoir wall comprises a vertical lower reservoir wall section 36 which meets the reservoir floor 32 and extends generally vertically upwards therefrom. Above the lower reservoir wall section 36 is a middle reservoir wall section 38. This extends upwardly from the lower reservoir wall section and is angled away from the vertical direction (i.e. inclined relative to the horizontal plane). The middle reservoir wall section 38, which may also be referred to as the angled reservoir wall section 38, is angled outwardly with respect to the reservoir with increasing distance above the reservoir floor (i.e. so the horizontal cross-section of the reservoir increases with increasing height above the reservoir floor 32). Above the angled (middle) reservoir wall section 38 is an upper reservoir wall section 40. This extends generally vertically upwards from the top of the angled reservoir wall section 38. Thus, at least a section 38 of the reservoir wall 32 is angled away from the vertical so as to be inclined with respect to a horizontal plane defined by a surface of cover media in the reservoir when the culture dish is in normal use. In this example the angled section 38 of the reservoir wall 34 extends all around the reservoir wall, but this might not necessarily be the case in all implementations.

The provision of the angled section 38 in the reservoir wall 34 can serve various purposes.

For example, the height of the join between the lower reservoir wall section 36 and the angled reservoir wall section 38 above the reservoir floor (i.e. the height of the lower reservoir wall section) may be selected according to a desired minimum level of cover media to be used when the culture dish is in normal use. The inventors have recognised that when filling a transparent culture dish with a transparent cover media it can in some circumstances be difficult for a user to reliably determine the height to which the cover media has been filled. This typically results in a user putting in more cover media than is necessary so the user feels certain a desired minimum level has been reached. This can be detrimental because not only is it wasteful to use more cover media than necessary, it gives rise to the potential for greater impact in the event of accidental spillage (since there will be more cover media to spill). The inventors have recognised the provision of a section of wall that is more angled relative to another section of wall allows a user to readily see when the depth of media in the reservoir passes the height of the change in wall angle. This is because the expanding perimeter of the media will be readily apparent as it extends over the angled section of wall. That is to say, a user can simply continue to fill the reservoir with cover media until the user sees the cover media starting to extend over the angled section of wall 38 (i.e. when the cover media starts to overflow the lower section of reservoir wall 36). At this stage the user knows the depth of cover media introduced into the reservoir is at least the height of the lower wall section. Thus, if the minimum depth of cover media (e.g. oil) for a given application is considered to be 1.5 mm, the height of the lower wall section 38 may be set at 1.5 mm. A user filling the reservoir 30 with oil will readily see when this height is exceeded as the oil starts to spread over the angled reservoir wall section 38.

This media-depth-indicating function of the angled section 38 of the reservoir wall 34 could be achieved without the angled section 38 of the reservoir wall 34 extending all around the reservoir 30. However, in addition to providing an indication of media depth, the angled reservoir wall section 38 also serves to in effect move the meniscus of the cover media (i.e. where the cover media meets the reservoir wall 34) further away from the wells than would otherwise be the case for a given depth and volume of cover media. This can be particularly advantageous in implementations in which the culture dish is intended for use in an incubator apparatus having time-lapse imaging functionality, for example an incubator apparatus 100 of the kind represented in FIG. 14. This is because the imaging systems for this type of incubator apparatus generally rely on optical paths that pass through the cover media (e.g. for imaging or illumination). It can therefore be important to reduce distortions that might otherwise be associated with curvature in the cover media associated with its meniscus. The provision of an angled section of reservoir wall, and in particular one which extends around the majority of the reservoir parameter, allows the cover media's meniscus to be further from the wells in the reservoir than would otherwise be the case for a given volume of cover media. Furthermore, the magnitude of the meniscus effect can be reduced by virtue of the cover media meeting the reservoir wall at an angle rather than square on.

In the particular example culture dish represented in FIGS. 2 to 13, and as schematically indicated in FIG. 9, the lower-reservoir wall 36 has a height of around 1.5 mm, the upper reservoir wall 40 has a height of around 2.5 mm, and the angled section of reservoir wall 38 extends over a vertical height of around 4 mm and is inclined at around 45 degrees to the horizontal plane. However, it will be appreciated the geometry of the reservoir may be different in different implementations. For example, the height of the lower-reservoir wall section 36 may be selected according to a desired minimum depth of cover media for the application at hand. Likewise, the inclination angle of the angle section 38 may be different in different implementations. For example, the angled section of the reservoir wall may be inclined with respect to the horizontal plane by an angle within a range selected from the group comprising: 10 degrees to 80 degrees; 20 degrees to 70 degrees; 30 degrees to 60 degrees; and 40 degrees to 50 degrees.

As noted above, the change in wall angle between the lower wall section 36 and the angled wall section 38 in effect provides a marker/visual cue that a user can readily see and use to identify when a level of cover media is greater than the height of the marker (because the periphery of the cover media surface will be seen to begin extending beyond the marker). In other examples there may be no vertical lower wall section 36—i.e. the angled section 38 of the reservoir wall may extend down to the reservoir floor. In this case a surface marker, for example a moulded line, may be provided in the angled surface at an appropriate height to in effect provide a marker for a minimum fill level. Thus, a user can recognise the desired level has been achieved when the surface of the cover media begins to extend beyond the marker provided on the angled wall section.

The upper wall section 40 extends above the level of the portion of the main body 24 of the culture dish 22 that surrounds the reservoir, for example as seen in FIG. 8. Thus, the upper wall section 40 in effect provides a vertically extending lip around the reservoir, for example to help avoid spillage. Furthermore, the upwardly-extending lip provided by the upper wall section 40 provides a seating for the lid 60 represented in FIG. 4.

The geometry of the interior of the lid 60 may be selected to broadly match the external extent of the upper wall section 40 in horizontal cross-section, and the height of the lid 60 may be selected to broadly correspond with the height of the upper wall section 40. Accordingly, the lid 60 can be readily located over the reservoir 30, as schematically represented in FIG. 4. In this example the lid 60 is further provided with an upwardly extending lip 62 around its perimeter. The inventors have found this naturally helps prevent users from moving their fingers across the surface of the lid 60 when handling the lid 62 and/or main body 24 of the culture dish 22. This can be especially advantageous if the culture dish is intended for use in an incubator apparatus having time-lapse imaging functionality. This is because the imaging systems in this type of incubator apparatus will generally rely on optical paths that pass through the lid 60 (e.g. for imaging or illumination), and so it can be important to reduce scattering and or shadowing that might occur from fingerprints or other marks on the lid 60.

Another significant aspect of the lid 60 as represented in the example of FIG. 4 is that it is arranged to be narrower than the culture dish. That is to say, when the lid 60 is in place it does not overhang the sides of the main body 24 of the culture dish 22. This means that if a user is holding the culture dish by its sides, there is a reduced risk of the user only gripping the lid 60 and allowing the main body 24 of the culture dish 22 to fall away. However, in other examples the lid may overhang the main body.

As noted above, the wells 42 in this example are provided at the bottom of a depression (trough) 44 in the reservoir floor 32. As can be seen in FIG. 10, for example, the trough (depression) 44 is defined by a trough floor (depression floor) 45 and a trough wall (depression wall) 47. In horizontal cross section the trough 44 has a generally arcuate form to accommodate the line of arc on which the wells 42 are arranged as discussed above. Thus, the trough wall 47 comprises two curved sides running azimuthally across the width of culture dish 22 and two shorter ends running broadly parallel to the sides of the culture dish 22, as shown in the figures.

The top of the trough in this example has a characteristic length along its curved sides of around 3.5 cm a characteristic width between its curved sides of around 0.7 cm. The trough 44 has a characteristic depth (from the top of the trough wall 47 to the trough floor 45) of around 0.5 cm. However, It will be appreciated the overall scale and exact geometry of the trough in horizontal cross-section is not of primary significance, and may be selected according to the implementation at hand, for example having regard to the arrangement of wells to be accommodated within the trough.

The trough wall 47 extends upwardly from the trough floor 45 and in this example comprises two sections, each of which extends all around the trough 44. Thus, the trough wall comprises an angled trough wall section 52 which meets the horizontal trough floor 45 (at a rounded corner) and extends generally away the trough floor 45 in a direction which is angled away from the vertical direction (i.e. inclined relative to the horizontal plane). The angled trough wall section 52, which may also be referred to as the lower trough wall section 52, is angled outwardly with respect to the trough with increasing distance above the trough floor (i.e. so the horizontal cross-section of the trough increases with increasing height above the trough floor 45). Above the angled trough wall section 52 is an upper trough wall section 53. The upper wall section 53 extends generally vertically upwards from the top of the angled trough wall section 52.

The trough 44 is provided so that it may be partially (or fully) filled with culturing media if there is a desire for the culturing media in the different wells 42 to be in fluid communication during culturing. That is to say, the individual wells 42 may in effect be filled to overflowing so the culturing media begins to fill the surrounding trough. Furthermore, the individual wells 42 may be simultaneously filled by simply introducing culturing media into the trough letting it flow into the wells. This will generally be faster and more convenient than filling the wells 42 individually. Because the trough has a horizontal cross-section which is less than the reservoir 30, it is possible for the culturing media in the different wells 42 to remain in fluid communication while using a smaller volume of culturing media than would otherwise be required (i.e. if it was necessary to partially fill the entire reservoir with culturing media to maintain the culturing media and different wells in fluid communication). FIG. 11 schematically shows a surface level 76 of culturing media filled to the top of the trough 44. However, in practice it can be expected the trough may only be partially filled. In this regard markings on the angled section of the trough wall may be provided to indicate when a particular culturing media fill level has been exceeded, for example in a manner similar to that described above for level of cover media in the reservoir. Likewise, in some examples the wall 47 of the trough 44 may be provided with an additional lower trough wall section below the angled trough wall section and which functionally corresponds to the lower reservoir wall section 36 discussed above for the reservoir 30 to provide an indication of when a particular level of culturing media is reached.

In addition to potentially being used to help a user identify a fill level for culturing media, and as for the angled section 38 of the reservoir wall, the angled section 52 in the trough wall 47 reduces the amount of media required to reach a certain media level.

In the particular example culture dish 22 represented in FIGS. 2 to 13, the upper trough wall section 53 has a height of around 2 mm, and the angled section of trough wall 52 extends over a vertical height of around 2 mm and is inclined at around 45 degrees to the horizontal plane. However, it will be appreciated the geometry of the trough may be different for different implementations. For example, the angled section 52 of the trough wall 47 may be inclined with respect to the horizontal plane by an angle within a range selected from the group comprising: 10 degrees to 80 degrees; 20 degrees to 70 degrees; 30 degrees to 60 degrees; and 40 degrees to 50 degrees.

To introduce culturing media into the wells a user will typically use a syringe or pipette. For example, a syringe or pipette preload with an appropriate volume of culturing media may be used and the culturing media simply squirted into the trough 45. To assist a user in this regard the trough wall 47 may be provided with a recess portion 49. This provides a region in the trough 44 into which the culturing media can be initially introduced, and in particular provides a region into which the culturing media may be introduced which is further away from the wells than would otherwise be the case. This can be advantageous, for example, if the wells already contain embryos, as it can help avoid disturbance to the embryos caused by fluid flows in the culturing media.

In addition to the trough 44 and wells 42, the reservoir 30 may also contain one or more flush reservoirs 46. These are openings in the floor of the reservoir, for example having a diameter and depth of around 3 mm. The flush reservoirs may be used to contain liquids used during the preparation of embryos for culturing, for example washing, in accordance with generally conventional techniques. In this regard, the flush reservoirs 46 may be used in a manner similar to the reservoirs 20 of the known culture dish 2 represented in FIG. 1. The flush reservoirs 46 comprise generally vertical walls, but with angled sections 48 at the base inclined relative to the horizontal plane. These angled sections can help locate objects in the flush reservoirs, for example by guiding objects (such as embryos being washed) towards the centre of the flush reservoir floor from where they may be more conveniently picked for removal from the flush reservoir 46, for example for transfer to one of the wells 42. Although not shown in the example culture dish 22 represented in FIGS. 2 to 13, in some example implementations the walls of the flush reservoirs 46 may be angled over a significant fraction of their vertical extent, either around their whole parameter, or in one or more specific directions to allow more ergonomic access (e.g. to allow a picking or dispensing pipette/syringe to reach the bottom of the reservoir when approaching from an increased angle away from vertical).

In addition to the above-described features within the reservoir 30 of the culture dish 22, there are various significant features of the culture dish outside the reservoir which contribute towards its convenience of use. For example, in some implementations the main body 24 of the culture dish 22 may be provided with what might be referred to as a note-taking region 50. This is a generally flat portion of the main body which is provided to allow a user to write on the culture dish, for example using a felt pen, to make annotations/notes relevant for the culture dish. For example, a user may wish to write a time at which a certain event relating to embryos being cultured occurred. However, it will be appreciated the specific nature of the information to be recorded by a user writing on the annotation area 50 is not significant. It is generally preferred for culture dishes of the kind described herein to comprise smooth surfaces to reduce the likelihood of contaminants becoming trapped. However, to facilitate writing on the annotation area, it may be provided with a surface texture which is different from, e.g. rougher than, the surface texture of other portions of the culture dish, for example portions that are more likely to come into contact with media and/or objects being cultured.

Another aspect of the culture dish 22 represented FIGS. 2 to 13 is the provision of a pair of protrusions/fins 26 towards one end of the culture dish (in this example the end that would be facing away from the centre of the rotating slide carrier 114 when the dish is located in an incubator apparatus 100 of the kind represented in FIG. 14. These fins 26 are provided to facilitate handling. The fins 26 are generally parallel to one another and extend vertically away from the main body 24 of the culture dish 22. The fins may, for example, have a size that is around 1 cm² in area (to broadly match the contact areas of a thumb and a fore finger), and a thickness of around 1 mm. In some respects the individual fins 26 may be similar to the fin 6 of the known slide design represented in FIG. 1. However, the provision of two handling fins 26, as opposed to a single handling fin, can in some circumstances be advantageous. For example, the two fins 26 allow a user to grip the culture dish with a thumb on the outside surface of one fin and a forefinger on the outside surface of another fin (i.e. using a pinch like grip). Having the two fins provides a larger handle to help provide the user with a more substantial/reliable grip.

Furthermore, the space between the fins 26 provides a convenient region 28 for labelling. That is to say, the culture dish 22 is provided with a labelling region 28 arranged on the main body between the pair of fins 26. The labelling region 28 may be used for receiving a label, for example an adhesive paper label, containing information relating to the culture dish. For example, the labelling may comprise identification information relating to the patient for which the embryos have been obtained, and/or culturing protocol information regarding the manner in which the embryos are to be cultured. This information may be recorded on the label in a general text format and/or in a machine-readable format, for example using a barcode or QR code format. Significantly, the provision of the labelling region 28 between the two fins 26 can help protect labelling in the labelling region from being damaged by a user's fingers as the culture dish 22 is being handled. In the example culture dish 2 represented in FIG. 1, adhesive labelling is typically applied to the region 8 adjacent the handling fin 6. This location for the labelling can result in a user's fingers and thumb damaging the representation of identification information on the label (e.g. by wearing it away or making it dirty) during handling. There is less chance of this happening with the double fin arrangement for the culture dish 22 represented in FIGS. 2 to 13 because the user's finger/thumb would not normally come into contact with a label in the labelling region when handling using the fins 26.

The surface of the labelling region 28 may be inclined relative to the horizontal plane, for example by an angle of around 30 degrees or so, such as represented in FIG. 6. This inclination allows the labelling region to be conveniently seen from both above and from an end-on direction relative to the culture dish.

Thus, and as described above, there are various aspects of culture dishes provided in accordance with embodiments of the invention which helped to improve on existing designs. It will be appreciated that culture dishes in accordance with various embodiments of the invention may incorporate some or all of the above-identified features, either alone or in various combinations. Furthermore, in accordance with certain embodiments of the invention, a culture dish may comprise additional features and/or variations of the features described above.

For example, whereas the wells 42 in the culture dish 22 described above are circularly symmetric about a vertical axis, in another example, the wells might not be circularly symmetric. For example, at least an upper portion of wells may be non-circular in horizontal cross-section. In particular, the openings in the reservoir floor defining the tops of the wells may be elongate, for example as schematically represented in FIG. 17. FIG. 17 shows in perspective cross-section a portion of a trough 144 of a culture dish 122 in accordance with another embodiment of the invention. Except as discussed herein, the culture dish 122 may follow the same general configuration as the culture dish 22 described above. However, in this example the trough 144 is slightly different from the trough 44 of the culture dish 22 represented in FIG. 10 in that it does not comprise an angled section of trough wall.

The wells 142 of the culture dish 122 represented in FIG. 17 each comprise an upper well section 154 and a lower well section 158 separated by a generally horizontal shelf section 156 (similar to the wells 42 of the culture dish 22 represented in FIGS. 2 to 13). The lower well section and the horizontal shell section of the wells 42 seen, for example, in FIG. 10 and the wells 142 seen in FIG. 17 are broadly the same. However, whereas the upper well section 54 seen in FIG. 10 is certainly symmetric, the upper well section 154 represented in FIG. 17 is elongate. In particular, the wells 142 have a greater extent in one direction (generally parallel to an axis of extent for the culture dish 122 in this example) than in another direction (generally parallel the width of the culture dish 122 in this example). This approach allows for a relatively shallow angle 156 to the upper section of the respective wells 142 to be provided along one direction, and this can assist a user is seeking to manipulate objects within the well 142 (since a user's manipulation tool can approach from a shallower angle than would otherwise be the case).

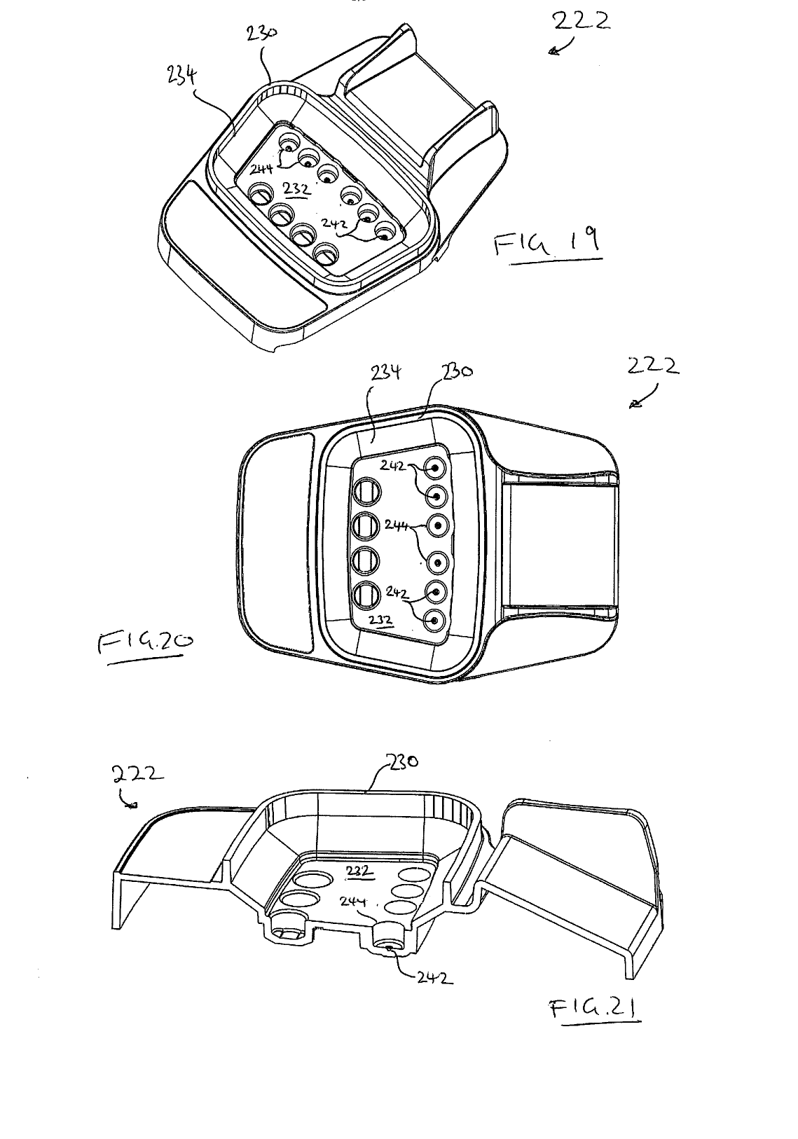

FIG. 18 schematically represents an additional feature that may be provided in the trough 44 of the culture dish 22 represented in FIGS. 2 to 13 in accordance with certain other embodiments of the invention. The additional feature is a raised section 150 of the trough floor 45 in the vicinity of the recess 49. For example, the raised section 150 may extend upward to provide a surface at a height of around 1 mm from the trough floor 45. During incubation of embryos the culturing media is sometimes replaced. This typically involves sucking a quantity of the culturing media out from underneath the cover media (e.g. oil), and then replacing it with an equivalent volume of new culturing media. As noted above, the region of the trough floor 45 adjacent the recess 49 can facilitate this process by providing a region into which the new culturing media can be introduced and from which existing culturing media can be removed. When removing culturing media it can be important not to remove too much to avoid the embryos accidentally coming into contact with the cover media. In addition, it can be important to avoid removing so much culture media that the cover media contact the floor of the trough. This is because there is a risk newly introduced culture media will form an intermediate layer in the cover media above the trough floor and so in effect be unavailable for the embryos. One might typically look to remove around 80% of the culturing media in a given media refresh cycle. However, it is often the case that a user will not remove as much as 80% of the culture media because of a fear of removing too much culturing media and damaging the embryos. However, in accordance with certain embodiments of the invention, the raised section 150 provides a platform on which the user may rest the nozzle of the tool used to remove the culturing media as it is removed. This prevents the user from removing an amount of culture media that would cause its level to drop below the level of the platform 150. Accordingly, a user can feel comfortable in removing the desired amount of culture media in the knowledge that if too much media is withdrawn, the excess will come from the oil covering layer, and not from the residual culturing media that is intended to remain in the culture dish. Accordingly, the provision of the raised section 150 allows for the convenient yet accurate removal of a desired volume of culturing media.

As has already been explained, it will of course be appreciated the various example dimensions and geometric configurations described above may be modified in accordance with other embodiments of the invention. For example, the overall shape and size of a culture dish may be selected in accordance with an incubator apparatus in which the culture dish is to be stored. Furthermore, whereas the above described example has focused on an arcuate line of 16 wells, the number of wells and their spatial arrangement can of course be different for different implementations. For example, rather than a single arcuate line of wells, there may be multiple arcuate lines of wells on differing radii, or wells arranged in one or more straight lines. It will also be appreciated that whereas the above-described embodiments have focused on application of culturing dishes for incubating embryos, culturing dishes in accordance with other embodiments of the invention may be used for culturing other objects.

It will furthermore be appreciated that culture dishes according to other embodiments of the invention may incorporate some or all of the features of the culture dish 22 described above without some of the other features of the culture dish 22 described above. That is to say, it will be appreciated that various features of embodiments of the invention described above are independently beneficial and can be used separately from other ones of the various features of embodiments of the invention described above. For example, in accordance with some embodiments of the invention, a culture dish may be provided having wells with a design of the kind described above, for example incorporating a shelf section to help prevent particles from sinking to the bottom of the well and/or non-circular cross-section, but the culture dish may not include features relating to the angled section of the reservoir wall and/or the handling fins and/or label region and/or other features as described above. Similarly, in accordance with some embodiments a culture dish may be provided with handling fins of the kind described above, but without some or all of the various other features described above. In broad summary, it will be appreciated that embodiments of the invention may comprise any appropriate combinations of the features described above, and in particular features which are functionality independent of one another may be incorporated together or separately in different embodiments.

Furthermore, various features of the embodiments described above may not be present in some other embodiments. For example, the culture dish 22 described above comprises a reservoir 30 with wells 42 provided in a depression (trough) 44 in the reservoir floor 32. This allows the trough to be partially filled with embryo culturing media so the embryo culturing media surrounding each embryo in the different wells is in fluid communication. However, in some cases there may be a desire for embryos to be surrounded by embryo culturing media which is not in fluid communication with embryo culturing media surrounding other embryos (for examples to allow analysis of the culture media for specific embryos). Thus wells may be individually provided in the floor of a reservoir without a common trough. An example culture dish 222 in accordance with this configuration is schematically represented in FIGS. 19, 20 and 21.

FIGS. 19, 20 and 21 respectively correspond with FIGS. 2, 5 and 8 for the culture dish 22 described above (although FIG. 19 shows the culture dish 222 from a slightly different direction compared to the view of the culture dish 22 represented in FIG. 2). Several aspects of the design of the culture dish 222 represented in FIGS. 19, 20 and 21 are similar to, and will be understood from, the culture dish 22 described above and are not described again in the interests of brevity. However, the culture dish 222 represented in FIGS. 19 to 21 differs from the culture dish 22 represented in FIG. 2 to 13 in that it comprises fewer wells for containing objects to be cultured and the wells are not provided in a common depression in the floor of a reservoir (i.e. the culture dish 222 does not include a common trough of the kind described above).

Thus the culture dish 222 of FIGS. 19 to 21 comprises six wells 242 for receiving embryos for culturing. The six wells 242 are spatially arranged along a single line comprising an arc of a circle (as for the sixteen wells 42 of the culture dish 22 described above). This can again allow a monitoring station of an incubation apparatus, for example an imaging device, to be brought into alignment with different wells 242 by simply rotating a slide carrier on which the culture dish 222 is appropriately positioned. The individual wells 242 may be generally the same as the wells 42 described above for the culture dish 22, for example in terms of their characteristic size and shape. The culture dish 222 comprises a reservoir 230 defined by a reservoir wall 234 and a reservoir floor 232. The general function and purpose of the reservoir 230 is the same as described above for the reservoir 30. Furthermore, the reservoir wall 234 may be generally the same as that described above for the culture dish 22. The wells 242 are provided in the floor 232 of the reservoir 230. More particularly, in this example implementation the wells 242 are provided within individual depressions 244 provided in the reservoir floor 232. That is to say, whereas for the culture dish 22 represented in FIG. 2 the wells 42 are provided in a common depression (trough) 44, for the culture dish 222 represented in FIGS. 19 to 21, the respective wells 242 are each provided in their own individual depression 244. Various aspects of the design of the individual depressions 244 (for examples in terms of depth and the depression wall's vertical profile) may be similar to corresponding aspects of the trough 44 described above. In some respects the embodiment represented in FIG. 19 may be seen as a variation on the embodiment represented in FIG. 2 in which there are multiple troughs each containing a single well instead of a single trough containing multiple wells. This approach can allow the culturing media associated with each well to remain separate from the culturing media associate with other wells during incubation, but otherwise the use of the culture dish 222 may be broadly the same as for the culture dish 22 described above.

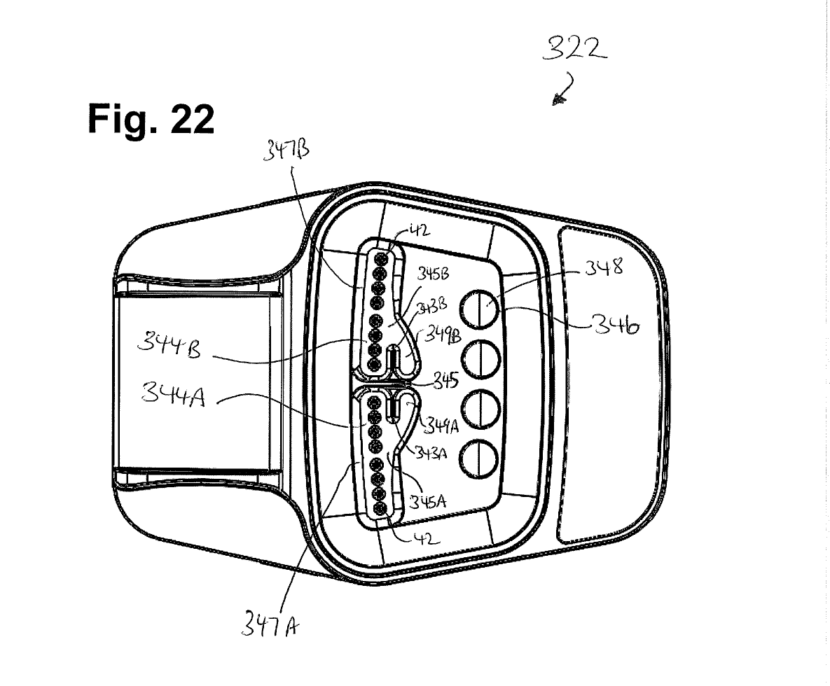

FIG. 22 schematically represents in plan view from above a culture dish 322 in accordance with another embodiment of the invention. The culture dish 322 of FIG. 22 is in most respects similar to, and will be understood from, the culture dish 22 discussed above and represented in plan view from above in FIG. 5, but with differences in respect of the depression (trough) in which the wells 42 are located. In particular, and as discussed above, the wells 42 in the culture dish 22 of FIG. 5 are all provided in a single depression (trough) 44 provided in the reservoir floor 32. However, as already noted above, in other examples the wells may be grouped together in a different troughs, and this is the approach adopted for the culture dish 322 of FIG. 22.

Thus the culture dish 322 represented in FIG. 22 may broadly be considered to correspond with the culture dish 22 of FIG. 5 but with the depression 44 of the culture dish 22 of FIG. 5 in effect divided into two depressions 344A, 344B for the culture dish 322 of FIG. 22. Thus, each depression 344A, 344B is respectively defined by a depression floor 345A, 345B and a depression wall 347A, 347B. In this regard, the combination of the two depressions 344A, 344B in FIG. 22 in effect correspond with the depression 44 of FIG. 5 but with an a dividing wall 345 extending across the depression around its centre (in a direction parallel to the dish's axis of extent). The dividing wall 345 in this example has a height that is a little higher (in this case 0.5 mm) than the depth of the depression so that it rises slightly above the floor of the reservoir. The dividing wall 345 in effected compartmentalises what would otherwise be a single depression into two separate depressions 344A, 344B. This has been found to be advantageous in some implementations since it can help reduce the magnitude of waves/bulk fluid motion in liquid media in the depression which might otherwise disturb embryos in the wells. Such effects may, for example, be created during manual handling of the culture dish 322, e.g. when outside an incubator, or during stopping and starting of motion of the culture dish 322 during its movement, e.g. rotation, in an incubator. In the example of FIG. 22, each depression compartment 344A, 344B contains 8 wells. However, it will be appreciated that in other configurations there may be a different number of depression compartments (i.e. multiple depressions corresponding to the depression 44 of FIG. 5 divided into more than two separate depressions may be provided) with different numbers of wells in the depressions.

Each of the depressions 344A, 344B comprises a recess portion 349A, 349B which together broadly corresponds to the recess portion 49 of the culture dish 22 of FIG. 5 but divided in two by the dividing wall 345 (i.e. the dividing wall 345 also compartmentalises the recess portion 49 of the example culture dish represented in FIG. 5 into two recess portions 349A, 349B in the example represented in FIG. 22). Furthermore, in the example culture dish 322 each recess portion 349A, 349B is (partially) separated from the remainder of its corresponding depression 344A, 344B by a respective protecting wall 343A, 343B extending away from the dividing wall. The protecting walls 343A, 343B do not extend all the way across their respective recess portions 349A, 349B so the recess portions 349A, 349B remain in fluid communication with the remainder of their respective depression compartments 344A, 344B. Thus fluid introduced into the respective recess portions, for example using a pipette, can flow around the protecting walls and into the parts of the depressions containing the wells. The function of the protecting walls 343A, 343B is to help deflect fluid introduced into the recess portion during filling away from the wells 42 adjacent the respective recess portions 349A, 349B to help reduce disturbance to embryos within the wells. In order to accommodate the protecting walls 343A, 343B, the width of the recess portions 349A, 349B in the direction parallel to the dividing wall 345 in the example culture dish 322 of FIG. 22 is slightly larger than the corresponding width of the recess portion in the example culture dish represented in FIG. 5.

Thus culture dishes for holding one or more objects to be cultured have been described. The culture dish has a main body comprising at least one well for receiving an object to be cultured and a quantity of culturing media for the object, such as a water-based growth media, and a reservoir for receiving a quantity of cover media, such as mineral oil. The at least one well is provided in a floor of the reservoir so that when in normal use cover media in the reservoir overlays culturing media in the at least one well. The reservoir is defined by the reservoir floor and a reservoir wall extending away from the reservoir floor. At least a section of the reservoir wall is angled away from the vertical so as to be inclined with respect to a horizontal plane defined by a surface of cover media in the reservoir when the culture dish is in normal use. The angled section of the reservoir wall can help reduce the appearance of a meniscus in the cover media overlaying the wells, and can furthermore be positioned so as to provide a ready indication of when the reservoir contains an appropriate level of cover media for culturing.

Further particular and preferred aspects of the present invention are set out in the accompanying independent and dependent claims. It will be appreciated that features of the dependent claims may be combined with features of the independent claims in combinations other than those explicitly set out in the claims.

### REFERENCES

[1] WO 09/003487 (Unisense Fetilitech A/S)
[2] WO 01/002539 (The Danish Institute of Agricultural Sciences)
[3] UK patent application GB 1401773.5 (Unisense Fetilitech A/S)
[4] UK patent applications GB 1401774.3 (Unisense Fetilitech A/S)

1. A culture dish for holding one or more object to be cultured, wherein the culture dish has a main body comprising:
at least one well for receiving an object to be cultured and a quantity of culturing media for the object;
and;
a reservoir for receiving a quantity of cover media, wherein the at least one well is provided in a floor of the reservoir so that when in normal use cover media in the reservoir overlays culturing media in the at least one well, wherein the reservoir is defined by the reservoir floor and a reservoir wall extending away from the reservoir floor, and wherein at least a section of the reservoir wall is angled away from the vertical so as to be inclined with respect to a horizontal plane defined by a surface of cover media in the reservoir when the culture dish is in normal use.

2. The culture dish of claim 1, wherein the angled section of the reservoir wall extends all around the reservoir.

3. The culture dish of claim 1, wherein the angled section of the reservoir wall is inclined with respect to the horizontal plane by an angle within a range selected from the group comprising: 10 degrees to 80 degrees; 20 degrees to 70 degrees; 30 degrees to 60 degrees; and 40 degrees to 50 degrees.

4. The culture dish of claim 1, wherein the angled section of the reservoir wall is located above a lower section of the reservoir wall, and wherein the angled section and the lower section are at different angles to the horizontal plane.

5. The culture dish of claim 4, wherein the height of the interface between the lower section of the reservoir wall and the angled section of the reservoir wall above the reservoir floor is selected according to a minimum level for cover media to be used when the culture dish is in normal use.

6. The culture dish of claim 1, wherein the angled section of the reservoir wall is located below an upper section of the reservoir wall, and wherein the angled section and the upper section are at different angles to the horizontal plane.

7. The culture dish of claim 6, wherein the upper section of the reservoir wall defines a section of the reservoir wall extending above a surface of the main body around the reservoir.

8. The culture dish of claim 1, further comprising a depression in the floor of the reservoir, wherein the depression is defined by a depression floor and a depression wall, and wherein the at least one well is provided in the depression floor.

9. The culture dish of claim 8, wherein at least a section of the depression wall is angled away from the vertical so as to be inclined with respect to the horizontal plane.

10. The culture dish of claim 9, wherein the angled section of the depression wall extends all around the depression.

11. The culture dish of claim 9, wherein the angled section of the depression wall is adjacent the depression floor at the bottom of the depression wall.

12. The culture dish of claim 9, wherein the angled section of the depression wall is inclined with respect to the horizontal plane by an angle within a range selected from the group comprising: 10 degrees to 80 degrees; 20 degrees to 70 degrees; 30 degrees to 60 degrees; and 40 degrees to 50 degrees.

13. The culture dish of claim 8, wherein the depression floor includes a raised section of floor that is higher than the surrounding depression floor.

14. The culture dish of claim 8, wherein a portion of the depression wall is recessed so that is further from the at least one well than parts of the depression wall adjacent the recessed portion.

15. The culture dish any of claim 14, further comprising a further wall portion arranged between the recessed portion of the of the depression wall and at least one of the at least one wells.

16. The culture dish of claim 8, wherein the at least one well comprises a plurality of wells provided in the depression floor of the depression.

17. The culture dish of claim 8, wherein the at least one well comprises a plurality of wells and the culture dish comprises at least one further depression in the floor of the reservoir, and wherein different wells are provided in different depressions.

18. The culture dish of claim 17, wherein the different depressions are separated from one another by one or more dividing wall.

19. The culture dish of claim 1, wherein the at least one well comprises an upper well section and a lower well section separated by a shelf section.

20. The culture dish of claim 1, wherein at least an upper portion of the at least one well is non-circular in horizontal cross-section.

21. The culture dish of claim 1, wherein the at least one well comprises a plurality of wells.

22. The culture dish of claim 1, further comprising a pair of fins extending away from the main body to provide a handle for the culture dish.

23. The culture dish of claim 22, further comprising a labelling region arranged on the main body between the pair of fins for receiving a label containing information relating to the culture dish.

24. The culture dish of claim 23, wherein the labelling region is inclined relative to the horizontal plane.

25. The culture dish of claim 1, further comprising a removable lid for covering the reservoir.

26. The culture dish of claim 25, wherein an upper surface of the removable lid comprises an upwardly extending lip at its periphery.

27. The culture dish of claim 25, wherein the lid does not overhang the main body when covering the reservoir.

28. The culture dish of claim 1, further comprising at least one receptacle for media to be used for preparing objects for culturing.

29. The culture dish of claim 28, wherein the at least one receptacle is within the reservoir.

30. The culture dish of claim 28, wherein at least a section of an inner wall of the least one receptacle is inclined with respect to the horizontal plane.

31. The culture dish of claim 1, further comprising an annotation area having a surface texture which is different from a surface texture of other parts of the culture dish.

32. The culture dish of claim 1, wherein the at least one well comprises a plurality of wells arranged along an arc of a circle.

33. The culture dish of 32, wherein the plurality of wells are spatially arranged in groups, wherein neighbouring wells that are in different groups are separated by a greater distance than neighbouring wells that are in the same groups.

34. An incubator apparatus comprising an incubation chamber comprising at least one culture dish according to claim 1.

35. A method of culturing at least one object, the method comprising:
providing a culture dish having a main body comprising:
at least one well for receiving an object to be cultured and a quantity of culturing media for the object; and; a reservoir for receiving a quantity of cover media, wherein the at least one well is provided in a floor of the reservoir so that when in normal use cover media in the reservoir overlays culturing media in the at least one well, wherein the reservoir is defined by the reservoir floor and a reservoir wall extending away from the reservoir floor, and wherein at least a section of the reservoir wall is angled away from the vertical so as to be inclined with respect to a horizontal plane defined by a surface of cover media in the reservoir when the culture dish is in normal use; and wherein the method further comprises
filling the reservoir with cover media to a level that meets the section of the reservoir wall is angled away from the vertical.

36.-37. (canceled)
